(19) United States
(12) Patent Application Publication
(10) Pub. No.: US 2006/0155294 A1
(43) Pub. Date: Jul. 13, 2006

(54) Tibial/femoral recutter with paddle

(75) Inventors: Scott Steffensmeier, Warsaw, IN (US); Adam Sanford, Warsaw, IN (US)

Correspondence Address: GREER, BURNS & CRAIN, LTD.;ATTN: JAMES K. FOLKER, 300 SOUTH WACKER DRIVE, SUITE 2500, CHICAGO, IL, 60606, US

(73) Assignee: Zimmer Technology, Inc.

(21) Appl. No.: 11/033,339

(22) Filed: Jan. 11, 2005

(51) Int. Cl. A61B 17/58

(52) U.S. Cl. 606/88

(57) ABSTRACT

The present invention relates to a bone-cutting guide used during arthroplasty. The bone-cutting guide is intended to be temporarily positioned against the distal femur or proximal tibia bone surface and to receive a cutting member. The bone-cutting guide is intended to cut the bone generally transverse to the length of the bone, and includes a main body and a paddle. The main body has a width defined between first and second surfaces. A cutting tool receiving portion, which is configured and arranged to receive the cutting member, extends from the first surface to the second surface. The main body is configured to be placed against a side surface of the bone. The paddle extends from the main body and includes a bone-contacting surface that is configured and arranged to be positioned generally parallel against the distal femur or the proximal tibia bone surface.

The present invention relates generally to a bone-cutting guide used during knee arthroplasty, where the bone-cutting guide is used for guiding a cutting member during cutting of the femur and/or the tibia. More particularly, the present invention relates to a bone-cutting guide configured to cut an initial cut or a recut on a femur and/or tibia. The concept of the present invention can be applied to many different types of arthroplasty, such as, for example, Unicompartmental Knee Arthroplasty (UKA) and Total Knee Arthroplasty (TKA).

Throughout this application various positional terms—such as distal, proximal, medial, lateral, anterior and posterior—will be used in the customary manner when referring to the human anatomy. More specifically, “distal” refers to the area away from the point of attachment to the body, while “proximal” refers to the area near the point of attachment the body. For example, the proximal femur refers to the portion of the femur near the hip, while the distal femur refers to the portion of the femur near the tibia. The terms “medial” and “lateral” are also essentially opposites, where “medial” refers to something situated closer to the middle of the body, while “lateral” refers to something situated closer to the left side or the right side of the body (than to the middle of the body). Finally, with regard to anterior and posterior, “anterior” refers to something situated closer to the front of the body and “posterior” refers to something situated closer to the rear of the body.

Also, the term “mechanical axis” of the femur refers to an imaginary line drawn from the center of the femoral head to the center of the distal femur at the knee and the term “anatomic axis” of the femur refers to an imaginary line drawn the middle of the femoral shaft (see FIGS. 4 and 7 for examples of the mechanical axis “m” and the anatomic axis “a”). The angle between the mechanical axis and the anatomic axis is generally approximately 6°.

The present invention provides an alternative approach to known methods and devices used for guiding the cutting blade for cutting the distal femur or the posterior femur during knee arthroplasty. One application of the cutting guide is to make an initial cut on the distal femur or proximal tibia. Another application of the cutting guide is to recut the initial bone cuts after resection of the distal femur or proximal tibia to accommodate an implant, for example. The present invention provides a minimally invasive and relatively uncomplicated tool that can be used to accurately cut or recut a bone surface.

### SUMMARY OF THE INVENTION

The present invention relates to a bone-cutting guide used during arthroplasty. The bone-cutting guide is intended to be temporarily positioned against the distal femur or proximal tibia bone surface and to receive a cutting member. The bone-cutting guide is intended to cut the bone generally transverse to the length of the bone, and includes a main body and a paddle. The main body has a width defined between first and second surfaces. A cutting tool receiving portion, which is configured and arranged to receive the cutting member, extends from the first surface to the second surface. The main body is configured to be placed against a side surface of the bone. The paddle extends from the main body and includes a bone-contacting surface that is configured and arranged to be positioned generally parallel against the distal femur or the proximal tibia bone surface.

More specifically, the present invention provides a bone-cutting guide intended to be used during arthroplasty, after resection of the femur or the tibia resulting in a resected femur surface or a resected tibia surface, for positioning a cutting member into proper orientation for recutting the femur or tibia. The cutting guide includes a main body and a cutting guide receiving portion, which is disposed on the main body. Further, the cutting guide receiving portion is configured and arranged to receive the cutting member. A paddle extends from the main body and includes a bone-contacting surface that is configured and arranged to be flushly positioned on the tibia or the femur.

Additionally, the present invention relates to a bone-cutting guide intended to be temporarily positioned against a first resected bone surface to receive a cutting member to cut a second resected bone surface. The cutting guide includes a main body having a cutting tool receiving portion configured and arranged to receive the cutting member. The main body is configured to be placed against a side surface of the bone. The cutting guide also includes at least one guide surface associated with the cutting tool receiving portion configured and arranged to guide the cutting member. A paddle extends from the main body and is fixed to the main body. The paddle includes a bone-contacting surface that is configured and arranged to be positioned generally parallel against the resected bone surface.

Another embodiment of the present invention relates to a bone-cutting guide intended to be temporarily positioned at one of a distal femur and a proximal tibia bone surface at a side surface of the bone and to receive a cutting member during arthroplasty to cut the bone generally transverse to the length of the bone. The cutting guide includes a main body defined between a front face and a back face, the main body having a cutting tool receiving portion extending from the front face to the back face and configured and arranged to receive the cutting member. The main body is configured to be placed against the side surface of the bone. The front surface of the cutting guide is contoured and configured to be positioned against one of a lateral femur portion and a medial proximal tibia portion and the back surface is contoured and configured to be positioned against one of a lateral tibia portion and a medial femur portion.

More specifically, a bone-cutting guide intended to be temporarily positioned adjacent a first resected bone surface at one of a first side surface and a second side surface, after resection with a resector, and to receive a cutting member to cut a second resected bone surface, is provided. The cutting guide includes a main body and an attachment arrangement configured to attach the body to the bone at one of the first and second side surfaces of the bone. The attachment arrangement is configured to receive at least one pin used to position the resector. A front surface of the main body is contoured and configured to be positioned at the first side surface of the bone, and a back surface opposite of the front surface is contoured and configured to be positioned at the second side surface of the bone.

### BRIEF DESCRIPTION OF THE SEVERAL VIEWS OF THE DRAWINGS

Preferred embodiments of the present invention are described herein with reference to the drawings wherein:

FIG. 1 is a top perspective view of a bone-cutting guide of the present invention;

FIG. 2 is a front view of the bone-cutting guide of FIG. 1;

FIG. 3 is a side view of a tibia and a femur with the bone-cutting guide of FIG. 1 positioned on the tibia;

FIG. 4 is a front view of a tibia and a femur with the bone-cutting guide of FIG. 1 positioned on the femur;

FIG. 5 is a perspective view of an alternate embodiment of a bone-cutting guide;

FIG. 6A is a front view of a prior art tibial resector;

FIG. 6B is a front view of the bone-cutting guide of FIG. 5;

FIG. 6C is a top view of a tibia and the bone-cutting guide of FIG. 5 being inserted onto pins placed by the tibial resector of FIG. 6A.

FIG. 7 is a front view of a tibia and a femur with the bone-cutting guide of FIG. 5 placed on four locations on the tibia and the femur;

FIG. 8 is a femoral top view of the bone-cutting guide of FIG. 5 placed at two locations on the femur; and

FIG. 9 is a tibial top view of the bone-cutting guide of FIG. 5 placed at two locations on the tibia.

### DETAILED DESCRIPTION OF THE INVENTION

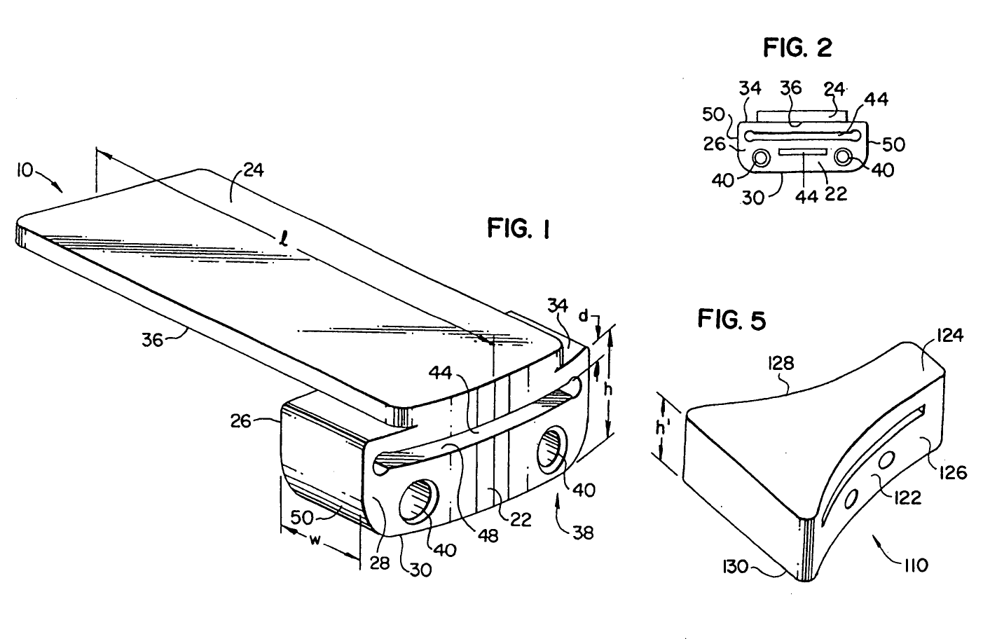

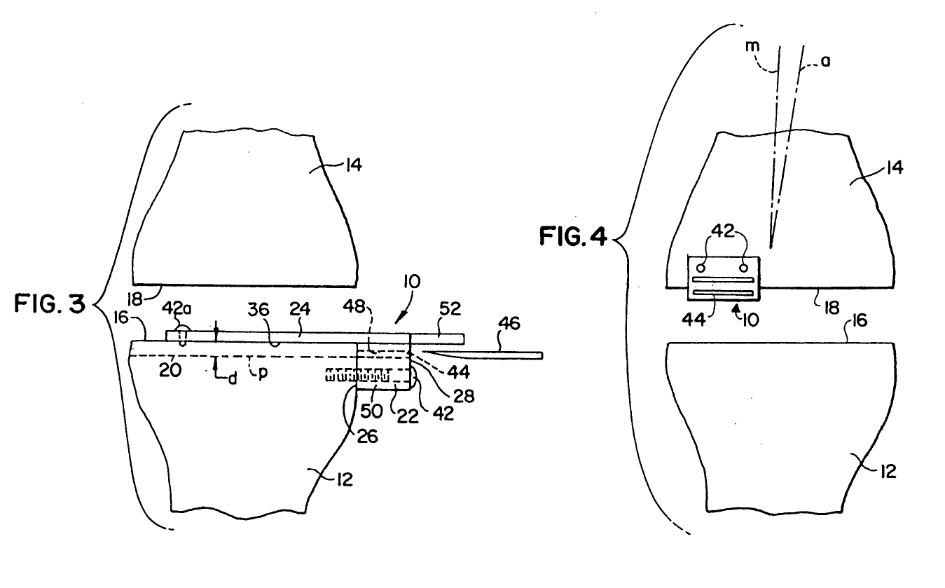

Turning to FIGS. 1-3, one embodiment of the present bone-cutting guide 10 will be shown and described, with FIG. 3 showing the bone-cutting guide in position between a tibia 12 and a femur 14. As shown in FIG. 3, the bone-cutting guide 10 is configured and arranged to be temporarily positioned upon a resected proximal surface 16 of a tibia 12, or on a resected distal surface 18 of a femur 14, to make a second resection surface 20 generally parallel to the initial resection surface 16 or 18. Generally, the bone-cutting guide 10 will be used on the distal femur 14 or the proximal tibia 12 to make a cut that is generally transverse to the length of the bone, where the length is measured along the mechanical axis “m”, the anatomical axis “a”, or any other length measurement. Although the present bone-cutting guide 10 is shown and described with respect to recutting the knee, it is contemplated that the bone-cutting guide can be used for any bone cutting procedure, including a primary cut, and on any bone.

Knee arthroplasty is the rebuilding of the knee, which can be done by resurfacing or relining the ends of bones where cartilage has worn away and bone has been destroyed. Arthroplasty also refers to total joint replacement, where all or part of an arthritic joint is removed and replaced with metal, ceramic, and/or plastic parts. Resection is the removal of part or all of a bone, such as, in this example, the tibia 12 or the femur 14. This is often done to improve function and relieve pain in the knee. Resection is performed by a surgeon, and often the initial resection surface 16, 18 must be corrected with the second resection surface 20 to remove sufficient bone to precisely accommodate an implant.

The bone-cutting guide 10 preferably includes two main components: a main body 22 and a paddle 24. As shown in FIG. 1, the main body 22 has a width “w” defined between a first, inner surface 26 and a second, outer surface 28, and a height “h” defined between the paddle 24 and a bottom surface 30. In the embodiment, the first, inner surface 26 is preferably substantially parallel to the second, outer surface 28 such that the width “w” is constant. For example, the width “w” of the main body 22 from the first, inner surface 26 to the second, outer surface 28 is preferably about 15 mm. Further, the first, inner surface 26 is preferably contoured so that it can be placed up against a peripheral side surface 32 of the bone to be resected. While the second, outer surface 28 is preferably contoured to be parallel with the first, inner surface 26, the outer surface can also form a linear plane, or any other desired shape.

The paddle 24 is preferably disposed on the main body 22 at a top surface 34 of the bone-cutting guide 10, and preferably extends generally perpendicularly with respect to the first and second surfaces 26, 28. However, it is contemplated that the paddle 24 can be at any angle with respect to the first and second surfaces 26, 28. In the preferred embodiment, the length of the paddle “l”, as measured from the second surface 28 to the end of paddle 24, is preferably about 50 mm to accommodate the smallest and the largest patient sizes, but any length is contemplated. Preferably, the paddle 24 is a thin, elongate member having a flat bone-contacting surface 36 that is configured and arranged to be positioned flush on a resected bone 12, 14. However, any shaped member which has a bone-contacting surface 36 configured to be positioned on the resected bone 12, 14 is contemplated. For example, the paddle 24 may have a contour for various applications, such as a primary cut. When the bone-contacting surface 36 is inserted and positioned on the resected bone in a generally flush engagement, the paddle 24 references the resection surface 16, 18 and the bone-cutting guide 10 alignment. In this position, the paddle 24 preferably extends along the initial resection surface 16, 18, and, depending on the size of the patient, can extend short of or beyond the mechanical and anatomic axes (“m” and “a”, respectively, of FIG. 3), and further, can extend substantially across the entire resection surface.

The paddle 24 is preferably integrally formed with the main body 22, but can also be detachable from the body. In an embodiment where the paddle 24 is detachable, it is contemplated that a plurality of different sized and shaped paddles can be used with a plurality of different sized and shaped bodies, depending on the anatomy of the patient.

As shown in FIG. 3, when the bone-contacting surface 36 of the paddle 24 is flushly engaged on the initial resected surface 16, the main body 22 is then preferably attached to the bone. FIG. 1 shows a top perspective view of a bone-cutting guide 10 including an attachment arrangement 38 configured for attaching the bone-cutting guide 10 to the bone. Preferably, the attachment arrangement 38 includes at least one, but preferably a plurality, of apertures 40 provided on the main body 22. Each aperture 40, if provided, is used with a fastener such as pin 42 of FIG. 3. Although a threaded version of pin 42 is shown in FIG. 3, a non-threaded pin can also be used. In use, the pin 42 is inserted through aperture 40 at the second, outer surface 28, to extend through the main body 22 and exit at the first, inner surface 26, such that it extends into the tibia 12 (or femur 14) to secure the bone-cutting guide 10 in position. It is also contemplated that other attachment arrangements 38 could be used to attach the bone-cutting guide 10 to the bone, such as protrusions on the first, inner surface 26 configured to be embedded in the bone, or by applying adhesive to the bone-cutting guide 10. Further, it is contemplated that the paddle 24 can also include an attachment arrangement 38 configured for attaching the paddle to the bone, such as with a pin 42a. Alternatively, the bone-cutting guide 10 can simply be held in place by the user.

The main body 22 of the bone-cutting guide 10 also includes a cutting tool receiving portion 44 for receiving a cutting member 46 to resect the bone. The cutting tool receiving portion 44 preferably comprises a slot in the main body 22 that is configured to receive and guide the cutting member 46, such as a blade shown in FIG. 3. Further, the cutting member 46 is preferably attached to a reciprocating or oscillating saw (not shown), or other cutting device configured for use during knee arthroplasty, or any other bone resection.

The cutting tool receiving portion 44 is preferably located a corrected distance “d” from the paddle 24. The corrected distance “d” is the distance from the initial resection surface 16, 18 to a location where the surgeon would like to make a second resected surface 20. In the preferred embodiment, the distance from the bone-contacting surface 36 of the paddle 24 to the cutting tool receiving portion 44 is two millimeters, however, other distances are also contemplated. Further, it is contemplated that a plurality of cutting tool receiving portions 44 corresponding to a series of corrected distances “d” may be disposed on the main body 22. For example, the main body 22 can have a plurality of cutting tool receiving portions 44 corresponding to corrected distances “d” of two millimeter increments. Further, the increments of corrected distances “d” may vary.

In the preferred embodiment, the cutting tool receiving portion 44 defines a cutting plane “p” (FIG. 3), which is generally perpendicular to the first surface 26 of the main body 22 and generally parallel to the bone-contacting surface 36 of the paddle 24. When the cutting plane “p” is generally parallel to the bone-contacting surface 36 of the paddle 24, the cutting plane “p” is also generally parallel to the initial resection 16, 18 of the bone.

In use, the cutting member 46 is inserted into the cutting tool receiving portion 44, and guided along at least one, but preferably a plurality of guide surfaces 48 in the cutting tool receiving portion 44, and a second resection surface 20 is made generally parallel with the initial resected surface 16, 18.

In certain instances, the surgeon may want to make the second resected surface 20 generally oblique to the initial resected surface 16, 18. In that instance, it is contemplated that the cutting plane “p” of the cutting tool receiving portion 44 would be made at the desired oblique angle, with respect to the bone-contacting surface 36 of the paddle 24. In the preferred embodiment including the slot as the cutting tool receiving portion 44, angling the slot to be oblique from the bone-contacting surface 36 of the paddle 24 would attain the oblique cutting plane “p”. An angled cutting tool receiving portion 44 on a bone cutting guide 10 can be used by a surgeon to alter the tibial or femoral slope, for example. Further, a set of cutting tools can be provided, with each one having a cutting tool receiving portion 44 at a different angle.

In addition, the cutting tool receiving portion 44 can be non-linear when a curved, resected surface 20 is required. Further, the cutting tool receiving portion 44 can have any orientation with respect to the main body 22, or any alignment relative to the paddle 24. Further still, in the preferred embodiment including the slot as the cutting guide receiving portion 44, the slot may extend to any surface on the main body 22, such as a side surface 50, to create a larger range of permissive movement of the cutting member 46.

Although the cutting tool receiving portion 44 is preferably a slot, it is also contemplated that other receiving portions can be incorporated. For example, an adjustable guide surface may be employed which retractably extends from the main body 22 (or the paddle 24), or is modularly added to the main body (or the paddle) to effect guide surfaces at different corrected distances “d”. In another contemplated embodiment, the bottom surface 30 of the main body forms the guide surface 48, or any other open slot could be used.

Referring again to FIG. 3, the bone-cutting guide 10 is engaged in the anterior tibia 12 with the paddle 24 disposed on the proximal surface 16. However, if the bone-cutting guide 10 is flipped over, the guide can be engaged in the anterior femur 14 with the paddle 24 disposed on the distal surface 18. Further, the bone-cutting guide 10 can be used on both the left and the right knees, and both the medial or lateral compartments of the knee, or in any other type of bone resection.

After the second resection surface 20 is cut, the at least one pin is preferably removed so that the bone-cutting guide 10 can be removed from the resected bone. A handle 52 can be disposed on the main body 22 and generally protrude perpendicularly from the second, outer surface 28 to facilitate the user in the entrance and exit of the bone-cutting guide 10.

Turning now to FIGS. 5-9, a second embodiment of the present bone-cutting guide 110 will be shown and described, with FIG. 7 showing the bone-cutting guide in position at two locations on the tibia 12 and two locations on the femur 14. Like features of the second embodiment 110 are numbered with similar reference numbers to the first embodiment 10. As shown in FIG. 7, the bone-cutting guide 110 is configured and arranged to be temporarily positioned adjacent a resected proximal surface 116 of a tibia 12, or adjacent a resected distal surface 118 of a femur 14, to make a second resection surface 120 generally parallel to the initial resection surface 116 or 118.

As shown in FIG. 5, the bone-cutting guide 110 preferably includes a main body 122 defined between a front surface 126 and a back surface 128. The main body also has a height “h′” defined between an upper surface 124 and a bottom surface 130. In the preferred embodiment, the front surface 126 is generally symmetrical to the back surface 128 to form a generally wedge-shaped body 122. Further, as seen in FIGS. 8 and 9, the front surface 126 is preferably contoured to be positioned against a first side surface 132 of the bone to be resected. Further still, the back surface 128 is preferably contoured to be positioned against a second side surface 134.

Referring now to FIGS. 7-9, the bone-cutting guide 110 is configured to be positioned at the least one side surface 132, and is preferably configured to be positioned at multiple side surfaces. More preferably, the bone-cutting guide 110 is contoured and configured to be positioned at the medial and lateral portions “Mt”, “Lt” of the proximal tibia 12 (FIG. 9), and the medial and the lateral portions “Mf”, “Lf” of the distal femur 14 (FIG. 8). While the contour of the bone-cutting guide 110 is generally flush with the lateral and medial portions “Lt”, “Mt” of the proximal tibia 12, it is an approximate fit to the lateral and medial portions “Lf”, “Mf” of the distal femur. However, even when used on the lateral and medial portions “Lf”, “Mf” of the femur, the contour of the bone-cutting guide 110 is an improvement over the linear surfaces of the prior art bone-cutting guides 110. Further, a single bone-cutting guide 110 can be used on all knee compartments.

In particular, the front surface 126 can be generally positioned against the lateral portion “Lf” of the distal femur 14 (FIG. 8). When this same guide 110 is rotated generally 180-degrees, the back surface 128 can be generally positioned against the medial portion “Mf” of the distal femur 14 (FIG. 8). When this same guide 110 is used on the tibia 12, the guide is flipped over about the upper surface 124 and the front surface 126 is generally positioned against the medial portion “Mt” of the tibia (FIG. 9). When the guide 110 is rotated generally 180-degrees, the back surface 128 can be generally positioned against the lateral portion “lt” of the tibia 12.

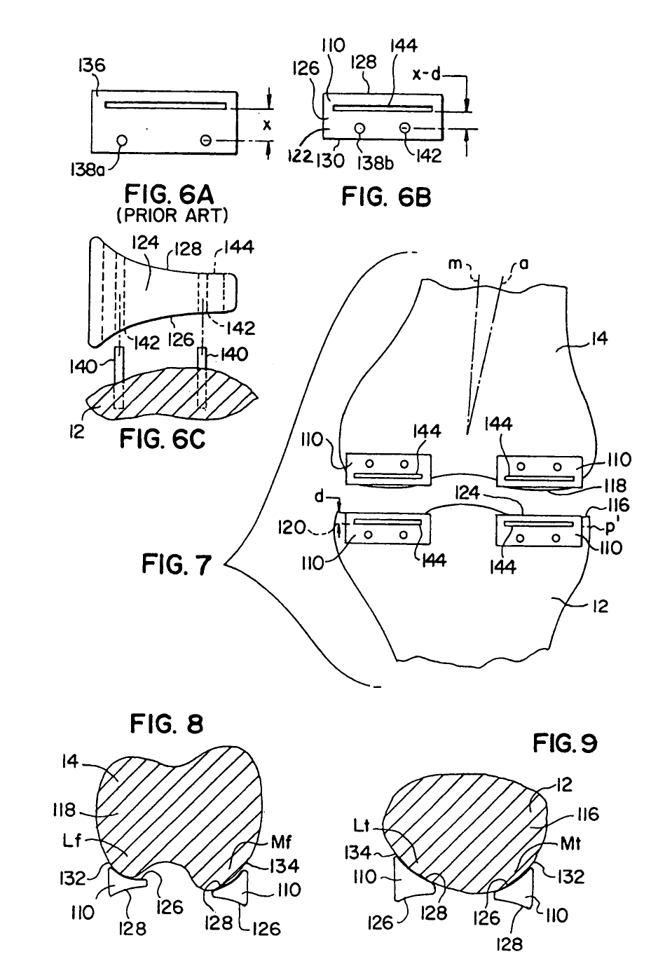

FIG. 6A shows a front view of a resector 136 (as is known in the art) having an attachment arrangement 138a configured for attaching the resector to the bone. FIG. 6B shows the bone-cutting guide 110 including an attachment arrangement 138b configured for attaching the bone-cutting guide 110 to the bone. Preferably, the attachment arrangements 138a and 138b are configured to receive pins 140 to attach the resector 136 and the cutting guide 110 to the bone. More preferably, the attachment arrangement 138a of the resector 136 receives pins 140, and after use of the resector, the resector is slid off and removed from the pins while the pins remain in the bone. When the bone-cutting guide 110 is placed into position adjacent the bone, the attachment arrangement 138b receives the pins 140 in at least one aperture 142 extending from the front surface 126 to the back surface 128.

Referring now to FIGS. 6B-6C, the main body 122 of the bone-cutting guide 110 also includes a cutting tool receiving portion 144 for receiving the cutting member 46 (FIG. 3) to resect the bone. The cutting tool receiving portion 144 also preferably comprises a slot in the main body 122 that is configured to receive and guide the cutting member 46.

The cutting tool receiving portion 144 is preferably located a distance “x-d” from the attachment arrangement 138b. If a distance “x” is the distance between the attachment arrangement 138a and the slot of the resector 136, the distance “x-d” between the attachment arrangement 138b and the slot 144 provides a corrected distance “d” (shown in FIG. 3), where the corrected distance “d” is the distance from the initial resection surface 116, 118 to the location where the surgeon desires to make the second resection surface 120. It is contemplated that a plurality of cutting tool receiving portions 144 corresponding to a series of corrected distances “d” may be disposed on the main body 122.

As is shown in FIGS. 3 and 7, the cutting tool receiving portion 144 defines a cutting plane “p′” (FIG. 7), which is generally parallel to the top surface 124 of the main body 122. The cutting plane “p′” is also generally parallel to the initial resection 116, 118 of the bone.

In certain instances, the surgeon may want to make the second resected surface 120 generally oblique to the initial resected surface 116, 118, which can be done as described with respect to the bone-cutting guide 10. In addition, the cutting tool receiving portion 144 can be non-linear, and can extend to any surface on the main body 122. Further, while the cutting tool receiving portion 144 is preferably a slot, it is also contemplated that other receiving portions can be incorporated, as described with respect to the first embodiment. Further, the bone-cutting guide 110 can be used on both the left and the right knees, and both the medial or lateral compartments of the knee, or in any other type of bone resection.

While various embodiments of the present invention have been shown and described, it should be understood that other modifications, substitutions and alternatives may be apparent to one of ordinary skill in the art. Such modifications, substitutions and alternatives can be made without departing from the spirit and scope of the invention, which should be determined from the appended claims.

Various features of the invention are set forth in the appended claims.

What is claimed is:

1. A bone-cutting guide intended to be temporarily positioned against one of a distal femur and a proximal tibia bone surface and to receive a cutting member during arthroplasty to cut the bone generally transverse to the length of the bone, the bone-cutting guide comprising:
a main body having a width defined between first and second surfaces, said main body having a cutting tool receiving portion extending from said first surface to said second surface and configured and arranged to receive the cutting member, said main body configured to be placed against a side surface of the bone; and
a paddle extending from said main body, said paddle having a bone-contacting surface that is configured and arranged to be positioned against one of said distal femur surface and said proximal tibia bone surface.

2. The bone-cutting guide according to claim 1, wherein said paddle extends generally perpendicularly from said main body.

3. The bone-cutting guide according to claim 1, wherein said cutting tool receiving portion forms a cutting plane that is generally parallel to said bone-contacting surface of said paddle.

4. The bone-cutting guide according to claim 1, wherein said cutting tool receiving portion forms a cutting plane that is generally oblique with respect to said bone contacting surface of said paddle.

5. The bone-cutting guide according to claim 1, wherein said cutting tool receiving portion has a plurality of slots.

6. The bone-cutting guide according to claim 1, wherein said cutting tool receiving portion is disposed on said main body a corrected distance from said paddle.

7. The bone-cutting guide according to claim 6, wherein said corrected distance from said paddle is selected from a plurality of cutting tool receiving portions.

8. The bone-cutting guide according to claim 1 further comprising an attachment arrangement configured to attach the bone-cutting guide to the bone.

9. The bone-cutting guide according to claim 8, wherein said attachment arrangement comprises at least one aperture disposed on said main body, wherein said at least one aperture extends from said first surface to said second surface and is configured and arranged to receive a pin.

10. The bone-cutting guide according to claim 1, wherein said first surface is generally contoured to correspond to the peripheral surface of the bone.

11. The bone-cutting guide according to claim 1 further comprising a handle extending from said second surface of said main body.

12. The bone-cutting guide according to claim 1, wherein the cutting tool receiving portion is configured to resect a distal femur to receive a femoral prosthesis in conjunction with knee-replacement surgery.

13. The bone-cutting guide according to claim 1, wherein the cutting tool receiving portion is configured to resect a proximal tibia to receive a tibial prosthesis in conjunction with knee-replacement surgery.

14. A bone-cutting guide intended to be used during arthroplasty, after resection of one of a distal femur and a proximal tibia resulting in one of a resected femur surface and a resected tibia surface, for positioning a cutting member into proper orientation for recutting one of the femur and the tibia, said cutting guide comprising:
a main body;
a cutting tool receiving portion disposed on said main body and configured and arranged to receive the cutting member; and
a paddle extending from said main body, said paddle including a bone-contacting surface that is configured and arranged to be flushly positioned on one of the resected femur surface and the resected tibia surface.

15. The bone cutting guide of claim 14, wherein said paddle extends generally perpendicularly from said main body.

16. The bone-cutting guide of claim 14, wherein said cutting guide receiving portion is a slot extending through said main body from a first surface to a second surface, said slot being parallel to said paddle.

17. The bone-cutting guide of claim 14 further comprising at least one guide surface on said cutting guide receiving portion configured and arranged to be engaged by the cutting member.

18. A bone-cutting guide intended to be temporarily positioned against a first resected bone surface and to receive a cutting member to cut a second resected bone surface, the bone-cutting guide comprising:
a main body having a cutting tool receiving portion configured and arranged to receive the cutting member, said main body configured to be placed against a side surface of the bone;
at least one guide surface associated with said cutting tool receiving portion configured and arranged to guide the cutting member; and
a paddle extending from said main body, said paddle including a bone-contacting surface that is configured and arranged to be positioned generally parallel against the first resected bone surface;
wherein said paddle and said body are fixed to each other.

19. The bone cutting guide of claim 18, wherein said paddle extends generally perpendicularly from said main body.

20. The bone-cutting guide according to claim 18, wherein said cutting tool receiving portion forms a cutting plane that is generally parallel to said bone-contacting surface of said paddle.

21. A bone-cutting guide intended to be temporarily positioned at one of a distal femur and a proximal tibia bone surface at a side surface of the bone and to receive a cutting member during arthroplasty to cut the bone generally transverse to the length of the bone, the bone-cutting guide comprising:
a main body defined between a front face and a back face, said main body having a cutting tool receiving portion extending from said front face to said back face and configured and arranged to receive the cutting member, said main body configured to be placed against the side surface of the bone;
wherein said front surface is contoured and configured to be positioned against one of a lateral femur portion and a medial proximal tibia portion and said back surface is contoured and configured to be positioned against one of a lateral tibia portion and a medial femur portion.

22. The bone-cutting guide according to claim 21 further comprising an attachment arrangement configured to attach the bone-cutting guide to the bone.

23. The bone-cutting guide according to claim 22 wherein said attachment arrangement comprises at least one aperture disposed on said main body, wherein said at least one aperture extends from said front surface to said back surface and is configured and arranged to receive a pin.

24. The bone-cutting guide according to claim 23 wherein said pin is preset by a resector, and said attachment arrangement is configured to slide onto said preset pin.

25. The bone-cutting guide according to claim 21 wherein said main body is generally wedge-shaped.

26. A bone-cutting guide intended to be temporarily positioned adjacent a first resected bone surface at one of a first side surface and a second side surface, after resection with a resector, and to receive a cutting member to cut a second resected bone surface, the bone cutting guide comprising:
a main body;
an attachment arrangement configured to attach said body to the bone at one of the first and second side surfaces of the bone, wherein said attachment arrangement is configured to receive at least one pin used to position the resector;
a front surface of said main body contoured and configured to be positioned at the first side surface of the bone; and
a back surface opposite of said front surface contoured and configured to be positioned at the second side surface of the bone.

27. The bone-cutting guide according to claim 26 wherein said front surface and said back surface are generally symmetrical.

28. The bone-cutting guide according to claim 26 wherein said front surface is contoured and configured to be positioned at a medial tibia portion and a lateral femur portion

29. The bone-cutting guide according to claim 26 wherein said back surface is contoured and configured to be positioned generally flush at a lateral tibia portion and a medial femur portion

30. The bone-cutting guide according to claim 26 wherein said front surface and said back surface are generally contoured to correspond to one of a lateral and medial tibia portion.
